(19) United States
(12) Patent Application Publication
(10) Pub. No.: US 2021/0137428 A1
(43) Pub. Date: May 13, 2021

(54) DIABETES MANAGEMENT SYSTEMS AND METHODS

(71) Applicant: Senseonics, Incorporated, Germantown, MD (US)

(72) Inventor: Barbara Montgomery, Gaithersburg, MD (US)

(73) Assignee: Senseonics, Incorporated, Germantown, MD (US)

(21) Appl. No.: 17/096,656

(22) Filed: Nov. 12, 2020

Related U.S. Application Data
(60) Provisional application No. 62/934,112, filed on Nov. 12, 2019.

(51) Int. Cl. A61B 5/145; A61B 5/00

(52) U.S. Cl. CPC A61B 5/14532; A61B 5/7475; A61B 5/742

(57) ABSTRACT

Systems and methods for determining an amount of time that a subject spends within a target analyte range by time-of-day. The systems and methods may determine a first time-in-target value indicating a percentage of time within a first time-of-day range that a subject's analyte level was less than a first threshold and greater than a second threshold using at least data points from first and second days. The systems and methods may determine a second time-in-target value indicating a percentage of time within a second time-of-day range that the subject's analyte level was less than the first threshold and greater than the second threshold using at least data points from the first and second days. The systems and methods may display the first time-in-target value and the second time-in-target value.

### CROSS-REFERENCE TO RELATED APPLICATION

The present application claims the benefit of priority to U.S. Provisional Application Ser. No. 62/934,112, filed on Nov. 12, 2019, which is incorporated herein by reference in its entirety.

### BACKGROUND

### Field of Invention

This disclosure relates to systems and methods for improved diabetes management. In particular, this disclosure relates to system and methods for determining an amount of time that a subject spends within a target analyte range by time-of-day.

### Discussion of Background

Diabetic patients experience variance in their blood-analyte levels. Spending too much time in a hyperglycemic or hypoglycemic state is associated with negative health outcomes. It is believed, therefore, that patients may benefit from systems that allow them to easily see the amount of time that they spend within a target analyte range, above the target analyte range, and/or below the target analyte range.

Moreover, patient routines may be relatively consistent from day-to-day within a given time-of-day range, and these routines may regularly cause deviations from the patient's target analyte range. Although these deviations may occur regularly (e.g., daily), the patient may be unaware of the relationship between her routine and the deviations from her target range, and the patient may therefore lack information needed to decide whether to adjust her routine within a time-of-day range.

Accordingly, there is a need for systems and methods to address these and other challenges, as described more fully below.

### SUMMARY

The following presents a simplified summary in order to provide a basic understanding of some aspects described herein. This summary is not an extensive overview of the claimed subject matter. It is intended to neither identify key or critical elements of the claimed subject matter nor delineate the scope thereof. Its sole purpose is to present some concepts in a simplified form as a prelude to the more detailed description that is presented later.

One aspect of the invention may provide a method including obtaining a plurality of data points. Each data point of the plurality of data points may indicate a subject's analyte level at a respective time. The plurality of data points may include at least first data points indicating the subject's analyte level at respective times within a first time-of-day range of a first day, second data points indicating the subject's analyte level at respective times within a second time-of-day range of the first day, third data points indicating the subject's analyte level at respective times within the first time-of-day range of a second day, and fourth data points indicating the subject's analyte level at respective times within the second time-of-day range of the second day. The first time-of-day range may be between a first time-of-day and a second time-of-day, the first time-of-day range is less than twenty-four hours. The second time-of-day range may be between a third time-of-day and a fourth time-of-day, and the second time-of-day range may be less than twenty-four hours. The first time-of-day range and the second time-of-day range may be different. The method may include determining a first time-in-target value indicating a percentage of time, within the first time-of-day range, that the subject's analyte level was less than a first threshold and greater than a second threshold using at least the first and third data points of the plurality of data points. The method may include determining a second time-in-target value indicating a percentage of time, within the second time-of-day range, that the subject's analyte level was less than the first threshold and greater than the second threshold using at least the second and fourth data points of the plurality of data points. The method may include displaying the first time-in-target value and the second time-in-target value.

In some aspects, the method may include determining a first time-above-target value indicating a percentage of time, within the first time-of-day range, that the subject's analyte level was greater than a third threshold using at least the first and third data points of the plurality of data points. The method may include determining a second time-above-target value indicating a percentage of time, within the second time-of-day range, that the subject's analyte level was greater than the third threshold using at least the second and fourth data points of the plurality of data points. The method may include displaying the first time-above-target value and the second time-above-target value. In some aspects, the third threshold may be equal to the second threshold. In some aspects, may method may further include determining a first time-of-day range time-just-above-target value indicating a percentage of time, within the first time-of-day range, that the subject's analyte level was less than the third threshold and greater than the first threshold using at least the first and third data points of the plurality of data points. In some aspects, the method may include determining a second time-of-day range time-just-above-target value indicating a percentage of time, within the second time-of-day range, that the subject's analyte level was less than the third threshold and greater than the first threshold using at least the second and fourth data points of the plurality of data points. In some aspects, the method may include displaying the first time-of-day range time-just-above-target value and the second time-of-day range time-just-above-target value.

In some aspects, the plurality of data points may include at least fifth data points indicating the subject's analyte level at respective times within a third time-of-day range of the first day and sixth data points indicating the subject's analyte level at respective times within the third time-of-day range of the second day, the third time-of-day range is between a fifth time-of-day and a sixth time-of-day, the third time-of-day range is less than twenty-four hours, the first time-of-day range, the second time-of-day range, and the third time-of-day range are different. The method may further include determining a third time-in-target value indicating a percentage of time, within the third time-of-day range, that the subject's analyte level was less than the first threshold and greater than the second threshold using at least the fifth and sixth data points of the plurality of data points. The method may include displaying the third time-in-target value.

In some aspects, the method may further include determining, based on the plurality of data points, an analyte trend line indicating the subject's analyte level over time. In some aspects, the method may further include partitioning the analyte trend line into a plurality of segments, each segment representing the subject's analyte level over a discrete period of time. The method may further include, for each respective segment, determining whether the segment indicates that the subject's analyte level was within a target range defined by the first threshold and the second threshold. Determining the first time-in-target value may include: determining a first total time represented by the segments within the first time-of-day range that indicate that the subject's analyte level was within the target range and comparing the first total time to a second total time representing the time elapsed within the first time-of-day range over at least the first day and the second day. In some aspects, the method may further include associating each respective segment with a tag indicating the segment's determined status relative to the first and second thresholds. In some aspects, the method may further include receiving user input to display a third time-in-target value indicating a percentage of time, within a third time-of-day range defined between a third time-of-day and a fourth time-of-day, that the subject's analyte level was within the target range, and the third time-of-day range may be user-defined and different than the first and second time-of-day ranges. In some aspects, the method may further include, in response to receiving the user input, determining the third time-in-target value by: determining a third total time represented by the segments within the third time-of-day range that indicate that the subject's analyte level was within the target range; and comparing the third total time to a fourth total time representing the time elapsed within the third time-of-day range over at least the first day and the second day. The method may further include displaying the third time-in-target value.

In some aspects, the first time-in-target value and the second time-in-target value may be two time-in-target values among a plurality of time-in-target values, each time-in-target value of the plurality of time-in-target values may indicate a respective percentage of time that the subject's analyte level was greater less than the first threshold and greater than the second threshold within a respective time-of-day range of a plurality of time-of-day ranges, and the plurality of time-in-target values may include comprising at least three time-in-target values. In some aspects, each of the plurality of time-of-day ranges may be non-overlapping and represents a time interval of eight hours or less. In some aspects, the plurality of time-of-day ranges may include a range representing a morning period, a range representing midday period, a range representing an evening period, and a range representing a night period.

In some aspects, the plurality of data points may be collected over a sample period comprising at least seven days, and each of the determined time-in-target values may reflect data collected from each day within the sample period. In some aspects, partitioning the analyte trend line into the plurality of segments may include: determining a first point at which the trend line exceeds the first threshold and a second point at which the trend line falls below the first threshold; and defining a segment as extending from the first point to the second point. In some aspects, partitioning the analyte trend line into the plurality of segments may include defining non-overlapping segments at regular time intervals.

In some aspects, the analyte may be glucose. In some aspects, the method may further include classifying the data points of the plurality of data points into at least at least a first group for data points indicating the subject's analyte level at respective times within the first time-of-day range and a second group for data points indicating the subject's analyte level at respective times within the second time-of-day range.

Another aspect of the invention may provide a system for monitoring analyte levels. The system may include a processor, a memory, and a display. The system may be configured to obtain a plurality of data points. Each data point of the plurality of data points may indicate a subject's analyte level at a respective time. The plurality of data points may include at least first data points may indicate the subject's analyte level at respective times within a first time-of-day range of a first day, second data points may indicate the subject's analyte level at respective times within a second time-of-day range of the first day, third data points may indicate the subject's analyte level at respective times within the first time-of-day range of a second day, and fourth data points may indicate the subject's analyte level at respective times within the second time-of-day range of the second day. The first time-of-day range may be between a first time-of-day and a second time-of-day, and the first time-of-day range may be less than twenty-four hours. The second time-of-day range may be between a third time-of-day and a fourth time-of-day, and the second time-of-day range may be less than twenty-four hours. The first time-of-day range and the second time-of-day range may be different. The system may be configured to determine a first time-in-target value indicating a percentage of time, within the first time-of-day range, that the subject's analyte level was less than a first threshold and greater than a second threshold using at least the first and third data points of the plurality of data points. The system may be configured to determine a second time-in-target value indicating a percentage of time, within the second time-of-day range, that the subject's analyte level was less than the first threshold and greater than the second threshold using at least the second and fourth data points of the plurality of data points. The system may be configured to display the first time-in-target value and the second time-in-target value.

In some aspects, the system may be further configured to determine a first time-above-target value indicating a percentage of time, within the first time-of-day range, that the subject's analyte level was greater than a third threshold using at least the first and third data points of the plurality of data points. The system may be further configured to determine a second time-above-target value indicating a percentage of time, within the second time-of-day range, that the subject's analyte level was greater than the third threshold using at least the second and fourth data points of the plurality of data points. The system may be further configured to display the first time-above-target value and the second time-above-target value. In some aspects, the third threshold may be equal to the second threshold. In some aspects, the system may be further configured to determine a first time-just-above-target value indicating a percentage of time, within the first time-of-day range, that the subject's analyte level was less than the third threshold and greater than the first threshold using at least the first and third data points of the plurality of data points. The system may be further configured to determine a second time-just-above-target value indicating a percentage of time, within the second time-of-day range, that the subject's analyte level was less than the third threshold and greater than the first threshold using at least the second and fourth data points of the plurality of data points. The system may be further configured to display the first time-just-above-target value and the second time-just-above-target value.

In some aspects, the plurality of data points may include at least fifth data points indicating the subject's analyte level at respective times within a third time-of-day range of the first day and sixth data points indicating the subject's analyte level at respective times within the third time-of-day range of the second day, the third time-of-day range may be between a fifth time-of-day and a sixth time-of-day, and the third time-of-day range may be less than twenty-four hours. The first time-of-day range, the second time-of-day range, and the third time-of-day range may be different, and the system may be further configured to: determine a third time-in-target value indicating a percentage of time, within the third time-of-day range, that the subject's analyte level was less than the first threshold and greater than the second threshold using at least the fifth and sixth data points of the plurality of data points; and display the third time-in-target value.

In some aspects, the system may be further configured to determine, based on the plurality of data points, an analyte trend line indicating the subject's analyte level over time. In some aspects, the system may be further configured to partition the analyte trend line into a plurality of segments, and each segment may represent the subject's analyte level over a discrete period of time. The system may be further configured to, for each respective segment, determine whether the segment indicates that the subject's analyte level was within a target range defined by the first threshold and the second threshold. Determining the first time-in-target value may include: determining a first total time represented by the segments within the first time-of-day range that indicate that the subject's analyte level was within the target range; and comparing the first total time to a second total time representing the time elapsed within the first time-of-day range over at least the first day and the second day.

In some aspects, the system may be further configured to associate each respective segment with a tag indicating the segment's determined status relative to the first and second thresholds. In some aspects, the system may be further configured to receive user input to display a third time-in-target value indicating a percentage of time, within a third time-of-day range defined between a third time-of-day and a fourth time-of-day, that the subject's analyte level was within the target range. The third time-of-day range may be user-defined and different than the first and second time-of-day ranges. The system may be further configured to, in response to receiving the user input, determine the third time-in-target value by: determining a third total time represented by the segments within the third time-of-day range that indicate that the subject's analyte level was within the target range; and comparing the third total time to a fourth total time representing the time elapsed within the third time-of-day range over at least the first day and the second day. The system may be further configured to display the third time-in-target value.

In some aspects, the first time-in-target value and the second time-in-target value may be two time-in-target values among a plurality of time-in-target values, each time-in-target value of the plurality of time-in-target values may indicate a respective percentage of time that the subject's analyte level was greater less than the first threshold and greater than the second threshold within a respective time-of-day range of a plurality of time-of-day ranges, and the plurality of time-in-target values may include at least three time-in-target values. In some aspects, each of the plurality of time-of-day ranges may be non-overlapping and represents a time interval of eight hours or less. In some aspects, the plurality of time-of-day ranges may include a range representing a morning period, a range representing midday period, a range representing an evening period, and a range representing a night period.

In some aspects, the plurality of data points may represent measurements collected over a sample period comprising at least seven days, and each of the determined time-in-target values reflects data may be collected from each day within the sample period. In some aspects, the step of partitioning the analyte trend line into a plurality of segments may include: determining a first point at which the trend line exceeds the first threshold and a second point at which the trend line falls below the first threshold; and defining a segment as extending from the first point to the second point. In some aspects, the step of partitioning the analyte trend line into a plurality of segments may include defining non-overlapping segments at regular time intervals.

In some aspects, the analyte may be glucose. In some aspects, the system may be further configured to classify the data points of the plurality of data points into at least at least a first group for data points indicating the subject's analyte level at respective times within the first time-of-day range and a second group for data points indicating the subject's analyte level at respective times within the second time-of-day range.

Further variations encompassed within the systems and methods are described in the detailed description of the invention below.

### BRIEF DESCRIPTION OF THE DRAWINGS

The accompanying drawings, which are incorporated herein and form part of the specification, illustrate various, non-limiting embodiments of the present invention. In the drawings, like reference numbers indicate identical or functionally similar elements.

FIG. 1 depicts an exemplary method for tracking a subject's analyte levels by time-of-day.

FIG. 2 depicts an exemplary method for determining a subject's time within a target range by time-of-day.

FIG. 3 depicts another exemplary method for determining a subject's time within a target range by time-of-day.

FIG. 4 depicts an exemplary time-in-range by time-of-day display.

FIG. 5 shows an exemplary time-in-range display.

FIG. 6 shows an exemplary analyte monitoring system.

FIG. 7 shows an exemplary display device.

FIG. 8 shows an exemplary processing system.

### DETAILED DESCRIPTION

While aspects of the subject matter of the present disclosure may be embodied in a variety of forms, the following description and accompanying drawings are merely intended to disclose some of these forms as specific examples of the subject matter. Accordingly, the subject matter of this disclosure is not intended to be limited to the forms or embodiments so described and illustrated.

FIG. 1 illustrates an exemplary method 100 for tracking a subject's analyte levels by time-of-day. The method 100 may be performed by a system (e.g., the analyte monitoring system 600 illustrated in FIG. 6) which may include, for example, one or more processors, one or more memories, and/or one or more displays. In some non-limiting embodiments, the method 100 may be performed by one or more of a transceiver 620, a display device 630, and a data management system 640 of the analyte monitoring system 600 illustrated in FIG. 6. In some embodiments, the method 100 may include a step 102 in which the system obtains a plurality of data points indicating a subject's analyte level at respective times. The plurality data points may have been collected over a sample period of multiple days. The sample period may be, for example and without limitation, two days, a week, a month, or any other suitable period of time.

In some embodiments, the plurality of data points may include first data points indicating the subject's analyte level at respective times within a first time-of-day range of a first day and second data points indicating the subject's analyte level at respective times within a second time-of-day range of the first day. In some embodiments, the plurality of data points may further include third data points indicating the subject's analyte level at respective times within the first time-of-day range of a second day and fourth data points indicating the subject's analyte level at respective times within the second time-of-day range of the second day. In some non-limiting embodiments, the first time-of-day range may be between a first time-of-day (e.g., 6 AM) and a second time-of-day (e.g., 12 PM). In some non-limiting embodiments, the first time-of-day range may be less than twenty-four hours (e.g., eight hours, six hours, four hours, three hours, two hours, or one hour). In some non-limiting embodiments, the second time-of-day range may be between a third time-of-day (e.g., 12 PM) and a fourth time-of-day (e.g., 6 PM). In some non-limiting embodiments, the second time-of-day range may be less than twenty-four hours (e.g., eight hours, six hours, four hours, three hours, two hours, or one hour). In some non-limiting embodiments, the first time-of-day range and the second time-of-day range may be different time-of-day ranges (e.g., non-overlapping time-of-day ranges). In some non-limiting embodiments, the second time-of-day range may start when the first time-of-day range ends. However, this is not required.

In some embodiments, the plurality of data points may be obtained, for example, using a continuous analyte monitoring system. In some embodiments, the plurality of data points may include measurements collected from each day within the sample period. In some embodiments, the measured analyte may be, for example and without limitation, glucose or oxygen.

In some embodiments, the method 100 may include a step 104 in which the system determines a first time-in-target value for the first time-of-day range. In some non-limiting embodiments, the first time-in-target value may indicate a percentage of time, within the first time-of-day range, that the subject's analyte level was within a target range defined between a first threshold and a second threshold. In some embodiments, the step 104 may be performed by analyzing at least data collected from within the first time-of-day range on the first day (e.g., the first data points) and data collected from within the first time-of-day range on the second day (e.g., the third data points).

In some embodiments, the method 100 may include a step 106 in which the system may determine a second time-in-target value for the second time-of-day range. The second time-in-target value may indicate a percentage of time, within the second time-of-day range, that the subject's analyte level was within the target range defined between the first threshold and the second threshold. In some embodiments, the step 106 may be performed by analyzing at least data collected from within the second time-of-day range on the first day (e.g., the second data points) and data collected from within the second time-of-day range on the second day (e.g., the fourth data points).

In some embodiments, the thresholds and/or time-of-day ranges may be selected and/or customized by an end user or caretaker (e.g., physician) or preselected by a manager of the system. In some embodiments, any desired number of thresholds and time-of-day ranges may be used. For example, although the embodiment above contemplated two thresholds representing a target range, other embodiments could use three, four, five, or more thresholds to allow differentiation between, for example, one or more of “high,” “very high,” “low,” and “very low” states. For example, the system may determine a first time-above-target value indicating a percentage of time, within the first time-of-day range, that the subject's analyte level was greater than a third threshold. The system may further determine a second time-above-target value indicating a percentage of time, within the second time-of-day range, that the subject's analyte level was greater than the third threshold. These values may indicate a percentage of time that the user is in a hyperglycemic state. By setting the third threshold above the first threshold, the system may determine whether the subject's analyte levels are above the third threshold (e.g., “very high”) or between the first threshold and the third threshold (e.g., “high” or “just-above-target”). Corresponding calculations may be used to determine a percentage of time that the subject's analyte levels are “low” or “very low,” based on comparisons to the second threshold (e.g., defining the boundary for a “low” status) and an optional fifth threshold (e.g., defining the boundary for a “very low” status). Any or all of the above-described values may be displayed, optionally in a single combined graphical display or in separate graphical displays.

In some embodiments, the user may define new thresholds retroactively. In such cases, the user may set thresholds defining the target range (and/or other ranges such as, for example and without limitation, one or more of high, very high, low, and very low), and the system may then calculate, using previously obtained data, one or more of time-in-target, time-just-above-target, time-above-target, time-just-below-target, and time-below-target values based on the new user-defined thresholds. In some embodiments, the system may calculate values based on current threshold information, receive new user-defined thresholds, and then re-calculate values based on the new thresholds. The same processes described herein could be used to perform these calculations based on new thresholds. That is, the processes may be performed once using first thresholds, and then after receiving user input defining new thresholds, the processes may be performed a second time using the new thresholds.

In some embodiments, any number of time-of-day ranges may be used. For example, percentage values may be calculated for each of four six-hour time-of-day ranges. In some embodiments, the six-hour time-of-day ranges may, for example and without limitation, reflect, respectively, analyte measurement data collected between (i) 12 AM to 6 AM, (ii) 6 AM to 12 PM, (iii) 12 PM to 6 PM, and (iv) 6 PM to 12 AM. In other embodiments, percentage values may be calculated for each of three eight-hour time-of-day ranges. In some embodiments, the eight-hour time-of-day ranges may, for example and without limitation, reflect, respectively, analyte measurement data collected between (i) 10 PM to 6 AM, (ii) 6 AM to 2 PM, and (iii) 2 PM to 10 PM. The sizes of the time-of-day ranges and the start- and end-points could be modified or shifted forward or backward as desired by an end user or system manager. In some embodiments, a plurality of time-of-day ranges may include a range representing a morning period, a range representing midday period, a range representing an evening period, and a range representing a night period.

In some embodiments where third (or more) time-of-day ranges are used, the method 100 may include an optional step 108 in which the system analyzes at least fifth and sixth data points which may respectively indicate the subject's analyte levels within a third time-of-day range on the first and second days. Based on these data points, the system may determine a third (or more) time-in-target values indicating a percentage of time, within the third time-of-day range, that the subject's analyte level was in the target range. In some non-limiting embodiments, the system may additionally or alternatively determine values indicating percentages of time, within the third time-of-day range, that the subject analyte level was just above, above, just below, and/or below the target range. In some non-limiting embodiments, in step 108, the system may additionally determine time-in-target values (and/or one or more of time-just-above, time-above, time-just-below, and time-below values) for any additional time-of-day ranges (e.g., fourth, fifth, and/or sixth time-of-day ranges).

In step 110, the system may display the first and second time-in-target values (and/or any additional time-in-target values and/or any time-just-above, time-above, time-just-below, and/or time-below values). For example, these values may be graphically displayed on a user's device (e.g., smartphone, tablet, computer).

In some embodiments, the user may set different time-of-day ranges for different periods of time or days of the week. For example, if a user knows that her weekend routine is different than her weekday routine, she may wish to set different time-of-day ranges to better track her behavioral patterns. As one example, a ‘morning’ time-of-day range could be scheduled to begin at 6 AM on weekdays and 8 AM on weekends. In some embodiments, a user may select a first set of time-of-day ranges for use on a first set of days (e.g., weekdays) and a second set of time-of-day ranges for use on a second set of days (e.g., weekends). In some embodiments, method 100 may be used to determine and display time-in-target values for the first set of time-of-day ranges using data collected during days within the first set of days (e.g., all weekdays within a three-month period), and method 100 may be repeated to determine and display time-in-target values for the second set of time-of-day ranges using data collected during days within the second set of days (e.g., all weekend days within the three-month period).

An exemplary graphical display of time-in-target values is shown in FIG. 4, which depicts an exemplary embodiment in which data collected over a one-month sample period is displayed using four time-of-day ranges. As reflected in FIG. 4, during the sample period, the subject spent 75% of the 12 AM-6 AM time-of-day range within her target analyte range, 8.5% of the 12 AM-6 AM time-of-day range within a high analyte range just above her target analyte range, and 16.4% of the 12 AM-6 AM time-of-day range below her target analyte range (including 11.4% of the 12 AM-6 AM time-of-day range within a low analyte range just below her target analyte range and 5% of the 12 AM-6 AM time-of-day range within a very low analyte range far below her target analyte range). For the 6 AM-12 PM time-of-day range, however, during the sample period, the subject spent 79.6% of the 6 AM-12 PM time-of-day range within her target analyte range, 14.6% of the 6 AM-12 PM time-of-day range above target (including 12.3% of the 6 AM-12 PM time-of-day range within a high analyte range just above her target analyte range and 2.3% of the 6 AM-12 PM time-of-day range within a very high analyte range far above her target analyte range), and 5.2% of the 6 AM-12 PM time-of-day range below her target analyte range. If a user sees that they consistently above- or below-target within a particular time-of-day range, she may adjust her behavior during that time frame to improve her analyte level targeting, for example, by modifying her eating, exercising, or insulin delivery behavior.

FIG. 5 shows the same data displayed in a single time-in-range chart for the sample period without breaking out the analyte information into time-of-day ranges. As is apparent from the chart, this data presentation permits monitoring of a user's overall effectiveness in maintaining her desired analyte range, but this presentation provides fewer insights for how the user might adjust her daily behavior to improve her amount of time within her target analyte range.

FIG. 2 shows an exemplary method 200 for determining a subject's time within a target range by time-of-day. In some embodiments, method 200 may include a step 202 in which the system determines an analyte trend line indicating the subject's analyte levels over time. In some embodiments, the trend line may be determined based on the plurality of data points. For example, the system may use neighboring data points to estimate a user's analyte levels in that period, optionally adjusting for data points that appear to be outliers, and/or interpolating between data points. In some embodiments, the method 200 may include a step 204 in which the system partitions the analyte trend line into a plurality of segments. In some embodiments, this partitioning step may be performed by partitioning the analyte trend line into a plurality of segments comprises defining non-overlapping segments at regular time intervals. For example, the trend line may be partitioned at regular intervals of, for example, 1 minute, 2 minutes, 5 minutes, 10 minutes, 20 minutes, or 60 minutes. In other embodiments, the segmentation may be selected based on the characteristics of the data. For example, a segment may be defined such that it represents a period of time that the trend line was above or below a certain threshold. Alternatively, a segment may be defined such that it represents a period of time within a given time-of-day range that the trend line was above or below a certain threshold. In some embodiments, segments may be determined by analyzing points of intersection between the trend line and the thresholds. For example, the system may determine a first point at which the trend line exceeds the first threshold and a second point at which the trend line falls below the first threshold, and define a segment as extending from the first point to the second point. Alternatively, the beginning and/or end of a time-of-day range may be used to define one or both endpoints of a segment. For example, if the user's analyte levels were within, above, or below a particular analyte level range for the entire time-of-day range, a segment may be defined to include that entire time-of-day range. Likewise, if the trend line exceeds a threshold within a time-of-day range and stays above the threshold, the intersection may define the start of a segment and the end of the time-of-day range may define the end of the segment.

In some embodiments, the method 200 may include a step 206 in which the system may determine, for each respective segment, whether the segment indicates that the subject's analyte level was within a target range defined by the first threshold and the second threshold. In some embodiments, the system may associate each respective segment with a tag indicating the segment's determined status relative to the first, second, third, etc. thresholds. For example, a segment may be tagged as representing an “in-target” interval, a “high” interval, a “very high” interval, a “low” interval, or a “very low” interval. Associating segments with tags may permit the system to quickly identify and display data in response to user queries. For example, if the user wishes to define a new time-of-day range, the system may quickly calculate statistics for the newly defined range by using the tags. In other embodiments, a user may search for all segments within a date-range that indicate a selected status, thus facilitating the user's identification of patterns in her analyte levels.

In some embodiments, the method 200 may include steps 208 and 210, which may be used to determine a time-in-target value, such as the first and/or second time-in-target values discussed above. In step 208, the system may determine a first total time represented by the segments within the first time-of-day range that indicate that the subject's analyte level was within the target range. For example, in some embodiments, the system may select each of the segments within the first time-of-day range that indicate that the subject's analyte level was within the target range, determine the time represented by each such segment, and then sum the respective time values. In step 210, the system may compare the first total time to a second total time representing the time elapsed within the first time-of-day range over at least the first day and the second day (e.g., over the sample period). In some embodiments, the system may calculate a percentage value by dividing the first total time by the second total time.

In some embodiments, a user may wish to see time-in-target (or time-above-target, time-below-target, etc.) statistics within a newly defined time-of-day range. For example, a user input a request to display a third time-in-target value indicating a percentage of time, within a third time-of-day range defined between a third time-of-day and a fourth time-of-day, that the subject's analyte level was within the target range. The third time-of-day range may be user-defined and different than the first and second time-of-day ranges. In some embodiments, in response to receiving the user input, the system may repeat steps 208 and 210 with respect to the new time-of-day range. For example, the system may determine the third time-in-target value by determining a third total time represented by the segments within the third time-of-day range that indicate that the subject's analyte level was within the target range. In some embodiments, the system may compare the third total time to a fourth total time representing the time elapsed within the third time-of-day range over at least the first day and the second day, and display the third time-in-target value.

FIG. 3 shows an alternative exemplary method 300 for determining a subject's time within a target range by time-of-day. In some embodiments, the method 300 may include an optional step 302 in which the system pre-processes the plurality of data points. For example, in some embodiments, the system may compare neighboring data points against one-another, against a trend line, or against a feasible biological range to determine whether one or more of the data points may be outliers, discarded, given lesser weight, or adjusted upwardly or downwardly.

In some embodiments, the method 300 may include a step 304 in which the system determines, for each respective data point, whether the data point indicates that the subject's analyte level was within a target range defined by the first threshold and the second threshold. In some embodiments, the system may associate each respective data point with a tag indicating the segment's determined status relative to the first, second, third, etc. thresholds. For example, a data point may be tagged as within an “in-target” interval, a “high” interval, a “very high” interval, a “low” interval, or a “very low” interval. Associating data points with tags may permit the system to quickly identify and display data in response to user queries. For example, if the user wishes to define a new time-of-day range, the system may quickly calculate statistics for the newly defined range by using the tags. In other embodiments, a user may search for all data points within a date-range that indicate a selected status, thus facilitating the user's identification of patterns in her analyte levels.

In some embodiments, the method may include a step 306 in which the system may determine a time interval associated with each respective data point. For example, if measurement data is recorded at regular intervals, each data point could represent a time-of-day range with a time interval corresponding to the measurement interval. In some embodiments, each data point may be associated with a time interval of 1 minute, 2 minutes, 3 minutes, 4 minutes, 5 minutes, 10 minutes, 15 minutes, 20 minutes, 30 minutes, or 1 hour. In some embodiments, the data point may have a timestamp indicating the effective time of the analyte measurement, and the data point's associated time interval may be defined such that the timestamp that is within the time interval. In some embodiments, the timestamp may be at the beginning or end of the time interval. In some embodiments, the timestamp may be at the center of the time interval. In some embodiments, time intervals may be defined such that the boundary from one interval to the next occurs at a midpoint between the timestamps of adjacent data points. For example, an interval for a first data point may be defined by selecting an interval start-time that is at a midpoint between the first data point and a second data point, and an interval end-time may be selected by determining a midpoint between the first data point and a third data point.

In some embodiments, the method 300 may include steps 308 and 310, which may be used to determine a time-in-target value, such as the first and/or second time-in-target values discussed above. In step 308, the system may determine a first total time represented by the data points within the first time-of-day range that indicate that the subject's analyte level was within the target range. For example, in some embodiments, the system may select each of the data points within the first time-of-day range that indicate that the subject's analyte level was within the target range, and sum the respective time interval values for each data point. In step 310, the system may compare the first total time to a second total time representing the time elapsed within the first time-of-day range over at least the first day and the second day (e.g., over the sample period). In some embodiments, the system may calculate a percentage value by dividing the first total time by the second total time. In some embodiments, data points may be collected at substantially regular intervals, thereby allowing a simple numerical count of data points to be used as a proxy for time represented by those data points. In these embodiments, steps 308 and 310 may be performed by determining a number of data points within the first time-of-day range that indicate that the subject's analyte level was within the target range and compare that value to a total number of data points within the first time-of-day range. Thus, the steps of determining and comparing total time 308, 310 may include embodiments where numbers of data points are used as proxies for elapsed time.

As discussed above, in some embodiments, a user may wish to see time-in-target (or time-above-target, time-below-target, etc.) statistics within a newly defined time-of-day range. For example, a user input a request to display a third time-in-target value indicating a percentage of time, within a third time-of-day range defined between a third time-of-day and a fourth time-of-day, that the subject's analyte level was within the target range. The third time-of-day range may be user-defined and different than the first and second time-of-day ranges. In some embodiments, in response to receiving the user input, the system may repeat steps 308 and 310 with respect to the new time-of-day range. For example, the system may determine the third time-in-target value by determining a third total time represented by the segments within the third time-of-day range that indicate that the subject's analyte level was within the target range. In some embodiments, the system may compare the third total time to a fourth total time representing the time elapsed within the third time-of-day range over at least the first day and the second day, and display the third time-in-target value.

FIG. 6 shows an exemplary analyte monitoring system 600 suitable for use with any of the methods described herein. The analyte monitoring system 600 may be a continuous analyte monitoring system (e.g., a continuous glucose monitoring system). In some embodiments, the analyte monitoring system 600 may include one or more of an analyte sensor 610, a transceiver 620, and a display device 630. In some embodiments, the analyte monitoring system 600 may additionally include a data management system (DMS) 640. In some embodiments, the DMS 640 may be a web-based DMS (e.g., hosted on a remote server). In some non-limiting embodiments, the DMS 640 may provide cloud storage for the analyte monitoring information of the system 600. In some embodiments, the analyte monitoring system 600 may additionally include one or more additional devices 650. In some non-limiting embodiments, the one or more additional devices 650 may include one or more wearable devices (e.g., one or more smart watches and/or one or more Fitbits), one or more infusion pumps, one or more analyte meters, and/or one or more additional devices.

In some embodiments, the sensor 610 may be small, fully subcutaneously implantable sensor. However, this is not required, and, in some alternative embodiments, the sensor 610 may be a partially implantable (e.g., transcutaneous) sensor or a fully external sensor. In some embodiments, the transceiver 620 may be an externally worn transceiver (e.g., attached via an armband, wristband, waistband, or adhesive patch). In some embodiments, the transceiver 620 may communicate with the sensor 610 to initiate and receive one or more sensor measurements via a wireless connection (e.g., via near field communication (NFC)) or a wired connection. In some embodiments, the sensor measurements may include one or more light measurements and/or one or more temperature measurements. In some embodiments, the one or more sensor measurements may be indicative of an amount or concentration of an analyte in a medium (e.g., interstitial fluid) of a living animal (e.g., a living human).

In some non-limiting embodiments, the transceiver 620 may calculate one or more analyte level (e.g., analyte concentrations) using at least the received sensor measurements. In some embodiments, the transceiver 620 may communicate information (e.g., one or more analyte levels) wirelessly (e.g., via a Bluetooth™ communication standard such as, for example and without limitation Bluetooth Low Energy) to a mobile medical application (MMA) running on a display device 630 (e.g., a smartphone or tablet). In some embodiments, the MMA may additionally or alternatively receive the information receive the information from the transceiver 620 through a wired connection (e.g., using a Universal Serial Bus (USB)) port. In some embodiments, the MMA may communicate with the data management system 640 (e.g., for plotting and sharing of the received information). In some embodiments, the MMA may additionally or alternatively communicate with one or more devices 650. In some embodiments, the methods described herein may be performed in part on in whole on any of the transceiver, the display device 630, the DMS 640, or on one or more paired devices 650. In some embodiments, the methods may be jointly performed across multiple nodes in the system.

FIG. 7 is a block diagram of a non-limiting embodiment of a display device 730, which may be the display device 630 of the analyte monitoring system 600. As shown in FIG. 7, in some embodiments, the display device 730 may include one or more of a connector 702, a connector integrated circuit (IC) 704, a charger IC 706, a battery 708, a computer 710, a first wireless communication IC 712, a memory 714, a second wireless communication IC 716, and a user interface 740.

In some embodiments in which the display device 730 includes the connector 702, the connector 702 may be, for example and without limitation, a Micro-Universal Serial Bus (USB) connector. The connector 702 may enable a wired connection to an external device, such as a personal computer or transceiver. The display device 730 may exchange data to and from the external device through the connector 702 and/or may receive power through the connector 702. In some embodiments, the connector IC 704 may be, for example and without limitation, a USB-IC, which may control transmission and receipt of data through the connector 702.

In some embodiments in which the display device includes the charger IC 706, the charger IC 706 may receive power via the connector 702 and charge the battery 708. In some non-limiting embodiments, the battery 708 may be, for example and without limitation, a lithium-polymer battery. In some embodiments, the battery 708 may be rechargeable, may have a short recharge duration, and/or may have a small size.

In some embodiments, the display device 730 may include one or more connectors and/or one or more connector ICs in addition to (or as an alternative to) connector 702 and connector IC 704. For example, in some alternative embodiments, the display device may include a spring-based connector (e.g., Pogo pin connector) in addition to (or as an alternative to) connector 702, and the display device 730 may use a connection established via the spring-based connector for wired communication to a personal computer or the transceiver and/or to receive power, which may be used, for example, to charge the battery 708.

In some embodiments in which the display device 730 includes the first wireless communication IC 712, the first wireless communication IC 712 may enable wireless communication with one or more external devices, such as, for example, one or more personal computers, one or more transceivers, one or more other display devices, and/or one or more devices (e.g., one or more wearable devices). In some non-limiting embodiments, the first wireless communication IC 712 may employ one or more wireless communication standards to wirelessly transmit data. The wireless communication standard employed may be any suitable wireless communication standard, such as an ANT standard, a Bluetooth standard, or a Bluetooth Low Energy (BLE) standard (e.g., BLE 4.0). In some non-limiting embodiments, the first wireless communication IC 712 may be configured to wirelessly transmit data at a frequency greater than 1 gigahertz (e.g., 2.4 or 5 GHz). In some embodiments, the first wireless communication IC 712 may include an antenna (e.g., a Bluetooth antenna). In some non-limiting embodiments, the antenna of the first wireless communication IC 712 may be entirely contained within a housing of the display device. However, this is not required, and, in alternative embodiments, all or a portion of the antenna of the first wireless communication IC 712 may be external to the display device housing.

In some embodiments, the display device 730 may include a transceiver interface device, which may enable communication by the display device with one or more transceivers. In some embodiments, the transceiver interface device may include the antenna of the first wireless communication IC 712 and/or the connector 702. In some non-limiting embodiments, the transceiver interface device may additionally or alternatively include the first wireless communication IC 712 and/or the connector IC 704.

In some embodiments in which the display device 730 includes the second wireless communication IC 716, the second wireless communication IC 716 may enable the display device to communicate with the DMS and/or one or more remote devices (e.g., smartphones, servers, and/or personal computers) via wireless local area networks (e.g., Wi-Fi), cellular networks, and/or the Internet. In some non-limiting embodiments, the second wireless communication IC 716 may employ one or more wireless communication standards to wirelessly transmit data. In some embodiments, the second wireless communication IC 716 may include one or more antennas (e.g., a Wi-Fi antenna and/or one or more cellular antennas). In some non-limiting embodiments, the one or more antennas of the second wireless communication IC 716 may be entirely contained within a housing of the display device. However, this is not required, and, in alternative embodiments, all or a portion of the one or more antennas of the second wireless communication IC 716 may be external to the display device housing.

In some embodiments in which the display device 730 includes the memory 714, the memory 714 may be non-volatile and/or capable of being electronically erased and/or rewritten. In some embodiments, the memory 714 may be, for example and without limitations a Flash memory.

In some embodiments in which the display device 730 includes the computer 710, the computer 710 may control the overall operation of the display device. For example, the computer 710 may control the connector IC 704, the first wireless communication IC 712, and/or the second wireless communication IC 716 to transmit data via wired or wireless communication. The computer 710 may additionally or alternatively control processing of received data (e.g., analyte monitoring data received from the transceiver).

In some embodiments in which the display device 730 includes the user interface 740, the user interface 740 may include one or more of a display 720 and a user input 722. In some embodiments, the display 720 may be a liquid crystal display (LCD) and/or light emitting diode (LED) display. In some non-limiting embodiments, the user input 722 may include one or more buttons, a keyboard, a keypad, and/or a touchscreen. In some embodiments, the computer 710 may control the display 720 to display data (e.g., analyte levels, analyte level rate of change information, alerts, alarms, and/or notifications). In some embodiments, the user interface 740 may include one or more of a speaker 724 (e.g., a beeper) and a vibration motor 726, which may be activated, for example, in the event that a condition (e.g., a hypoglycemic or hyperglycemic condition) is met.

In some embodiments, the computer 710 may execute a mobile medical application (MMA). In some embodiments, the display device 730 may receive analyte monitoring data from the transceiver 620. In some non-limiting embodiments, the received analyte monitoring data may include one or more analyte levels, one or more analyte level rates of change, and/or one or more sensor measurements. In some embodiments, the received analyte monitoring data may additionally or alternatively include alarms, alerts, and/or notifications. In some embodiments, the MMA may display some or all of the received analyte monitoring data on the display 720 of the display device. In some alternative embodiments, the received analyte monitoring data may include one or more sensor measurements and does not include analyte levels, and the display device 730 may calculate one or more analyte levels using the one or more sensors measurements. In some alternative embodiments, the received analyte monitoring data may include one or more analyte levels but does not include analyte level rates of change, and the display device 730 may calculate one or more analyte level rates of change using the one or more analyte levels. In some non-limiting alternative embodiments, the display device 730 may calculate one or more analyte levels and calculate one or more analyte level rates of change using at least the one or more analyte levels calculated by the display device 730.

In some embodiments, the analyte monitoring system may calibrate the conversion of raw sensor measurements to analyte levels (e.g., analyte concentrations). In some embodiments, the calibration may be performed approximately periodically (e.g., every 12 or 24 hours). In some embodiments, the calibration may be performed using one or more reference measurements (e.g., one or more self-monitoring blood glucose (SMBG) measurements). In some embodiments, the reference measurements may be entered into the analyte monitoring system using the user interface 740 of the display device. In some embodiments, the display device 730 may convey one or more references measurements to the transceiver 620, and the transceiver 620 may use the one or more received reference measurements to perform the calibration. In some alternative embodiments (e.g., embodiments in which the display device 730 calculates one or more analyte levels), the display device 730 may use the one or more received reference measurements to perform the calibration.

FIG. 8 is a block diagram of a non-limiting embodiment of the computer 710 of the analyte monitoring system. As shown in FIG. 8, in some embodiments, the computer 710 may include one or more processors 522 (e.g., a general purpose microprocessor) and/or one or more circuits, such as an application specific integrated circuit (ASIC), field-programmable gate arrays (FPGAs), a logic circuit, and the like. In some embodiments, the computer 710 may include a data storage system (DSS) 523. The DSS 523 may include one or more non-volatile storage devices and/or one or more volatile storage devices (e.g., random access memory (RAM)). In embodiments where the computer 710 includes a processor 522, the DSS 523 may include a computer program product (CPP) 524. CPP 524 may include or be a computer readable medium (CRM) 526. The CRM 526 may store a computer program (CP) 528 comprising computer readable instructions (CRI) 530. In some embodiments, the CRM 526 may store, among other programs, the MMA, and the CRI 530 may include one or more instructions of the MMA. The CRM 526 may be a non-transitory computer readable medium, such as, but not limited, to magnetic media (e.g., a hard disk), optical media (e.g., a DVD), solid state devices (e.g., random access memory (RAM) or flash memory), and the like. In some embodiments, the CRI 530 of computer program 528 may be configured such that when executed by processor 522, the CRI 530 causes the computer 710 to perform steps described below (e.g., steps described below with reference to the MMA). In other embodiments, the computer 710 may be configured to perform steps described herein without the need for a computer program. That is, for example, the computer 710 may consist merely of one or more ASICs. Hence, the features of the embodiments described herein may be implemented in hardware and/or software.

In some embodiments in which the user interface 740 of the display device includes the display 718, the MMA may cause the display device to provide a series of graphical control elements or widgets in the user interface 740, such as a graphical user interface (GUI), shown on the display 718. The MMA may, for example without limitation, cause the display device to display analyte related information in a GUI such as, but not limited to: one or more of analyte information, current analyte levels, past analyte levels, predicted analyte levels, user notifications, analyte status alerts and alarms, trend graphs, analyte level rate of change or trend arrows, and user-entered events. In some embodiments, the MMA may provide one or more graphical control elements that may allow a user to manipulate aspects of the one or more display screens. Although aspects of the MMA are illustrated and described in the context of glucose monitoring system embodiments, this is not required, and, in some alternative embodiments, the MMA may be employed in other types of analyte monitoring systems.

In some embodiments where the display device 630 communicates with a transceiver 620, which in turn obtains sensor measurement data from an analyte sensor 610, the MMA may cause the display device 630 to receive and display one or more of analyte data, trends, graphs, alarms, and alerts from the transceiver 620. In some embodiments, the MMA may store analyte level history and statistics for a patient on the display device (e.g., in memory 714 and/or DSS 533) and/or in a remote data storage system.

In some embodiments, a user of the display device 630, which may be the same or different individual as patient, may initiate the download of the MMA from a central repository over a wireless cellular network or packet-switched network, such as the Internet. Different versions of the MMA may be provided to work with different commercial operating systems, such as the Android OS or Apple OS running on commercial smart phones, tablets, and the like. For example, where display device 630 is an Apple iPhone, the user may cause the display device 630 to access the Apple iTunes store to download a MMA compatible with an Apple OS, whereas where the display device is an Android mobile device, the user may cause the display device 630 to access the Android App Store to download a MMA compatible with an Android OS.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

1. A method comprising:
obtaining a plurality of data points, wherein:
each data point of the plurality of data points indicates a subject's analyte level at a respective time,
the plurality of data points includes at least first data points indicating the subject's analyte level at respective times within a first time-of-day range of a first day, second data points indicating the subject's analyte level at respective times within a second time-of-day range of the first day, third data points indicating the subject's analyte level at respective times within the first time-of-day range of a second day, and fourth data points indicating the subject's analyte level at respective times within the second time-of-day range of the second day,
the first time-of-day range is between a first time-of-day and a second time-of-day, the first time-of-day range is less than twenty-four hours,
the second time-of-day range is between a third time-of-day and a fourth time-of-day, the second time-of-day range is less than twenty-four hours, and
the first time-of-day range and the second time-of-day range are different;
determining a first time-in-target value indicating a percentage of time, within the first time-of-day range, that the subject's analyte level was less than a first threshold and greater than a second threshold using at least the first and third data points of the plurality of data points;
determining a second time-in-target value indicating a percentage of time, within the second time-of-day range, that the subject's analyte level was less than the first threshold and greater than the second threshold using at least the second and fourth data points of the plurality of data points; and
displaying the first time-in-target value and the second time-in-target value.

2. The method of claim 1, further comprising:
determining a first time-above-target value indicating a percentage of time, within the first time-of-day range, that the subject's analyte level was greater than a third threshold using at least the first and third data points of the plurality of data points;
determining a second time-above-target value indicating a percentage of time, within the second time-of-day range, that the subject's analyte level was greater than the third threshold using at least the second and fourth data points of the plurality of data points; and
displaying the first time-above-target value and the second time-above-target value.

3. The method of claim 2, wherein the third threshold is equal to the second threshold.

4. The method of claim 2, further comprising:
determining a first time-of-day range time-just-above-target value indicating a percentage of time, within the first time-of-day range, that the subject's analyte level was less than the third threshold and greater than the first threshold using at least the first and third data points of the plurality of data points;
determining a second time-of-day range time-just-above-target value indicating a percentage of time, within the second time-of-day range, that the subject's analyte level was less than the third threshold and greater than the first threshold using at least the second and fourth data points of the plurality of data points; and
displaying the first time-of-day range time-just-above-target value and the second time-of-day range time-just-above-target value.

5. The method of claim 1, wherein the plurality of data points includes at least fifth data points indicating the subject's analyte level at respective times within a third time-of-day range of the first day and sixth data points indicating the subject's analyte level at respective times within the third time-of-day range of the second day, the third time-of-day range is between a fifth time-of-day and a sixth time-of-day, the third time-of-day range is less than twenty-four hours, the first time-of-day range, the second time-of-day range, and the third time-of-day range are different, and the method further comprises:
determining a third time-in-target value indicating a percentage of time, within the third time-of-day range, that the subject's analyte level was less than the first threshold and greater than the second threshold using at least the fifth and sixth data points of the plurality of data points; and
displaying the third time-in-target value.

6. The method of claim 1, further comprising determining, based on the plurality of data points, an analyte trend line indicating the subject's analyte level over time. The method of claim 6, further comprising:
partitioning the analyte trend line into a plurality of segments, each segment representing the subject's analyte level over a discrete period of time;
for each respective segment, determining whether the segment indicates that the subject's analyte level was within a target range defined by the first threshold and the second threshold;
wherein determining the first time-in-target value comprises:
determining a first total time represented by the segments within the first time-of-day range that indicate that the subject's analyte level was within the target range; and
comparing the first total time to a second total time representing the time elapsed within the first time-of-day range over at least the first day and the second day.

8. The method of claim 7, further comprising associating each respective segment with a tag indicating the segment's determined status relative to the first and second thresholds.

9. The method of claim 7, further comprising:
receiving user input to display a third time-in-target value indicating a percentage of time, within a third time-of-day range defined between a third time-of-day and a fourth time-of-day, that the subject's analyte level was within the target range, wherein the third time-of-day range is user-defined and different than the first and second time-of-day ranges;
in response to receiving the user input, determining the third time-in-target value by:
determining a third total time represented by the segments within the third time-of-day range that indicate that the subject's analyte level was within the target range; and
comparing the third total time to a fourth total time representing the time elapsed within the third time-of-day range over at least the first day and the second day; and
displaying the third time-in-target value.

10. The method of claim 1, wherein the first time-in-target value and the second time-in-target value are two time-in-target values among a plurality of time-in-target values, each time-in-target value of the plurality of time-in-target values indicating a respective percentage of time that the subject's analyte level was greater less than the first threshold and greater than the second threshold within a respective time-of-day range of a plurality of time-of-day ranges, and the plurality of time-in-target values comprising at least three time-in-target values.

11. The method of claim 10, wherein each of the plurality of time-of-day ranges is non-overlapping and represents a time interval of eight hours or less.

12. The method of claim 10, wherein the plurality of time-of-day ranges comprises a range representing a morning period, a range representing midday period, a range representing an evening period, and a range representing a night period.

13. The method of claim 1, wherein the plurality of data points are collected over a sample period comprising at least seven days, and each of the determined time-in-target values reflects data collected from each day within the sample period.

14. The method of claim 7, wherein partitioning the analyte trend line into the plurality of segments comprises:
determining a first point at which the trend line exceeds the first threshold and a second point at which the trend line falls below the first threshold; and
defining a segment as extending from the first point to the second point.

15. The method of claim 7, wherein partitioning the analyte trend line into the plurality of segments comprises defining non-overlapping segments at regular time intervals.

16. The method of claim 1, wherein the analyte is glucose.

17. The method of claim 1, further comprising classifying the data points of the plurality of data points into at least at least a first group for data points indicating the subject's analyte level at respective times within the first time-of-day range and a second group for data points indicating the subject's analyte level at respective times within the second time-of-day range.

18. A system for monitoring analyte levels, the system comprising:
a processor;
a memory; and
a display;
wherein the system is configured to:
obtain a plurality of data points, wherein:
each data point of the plurality of data points indicates a subject's analyte level at a respective time,
the plurality of data points includes at least first data points indicating the subject's analyte level at respective times within a first time-of-day range of a first day, second data points indicating the subject's analyte level at respective times within a second time-of-day range of the first day, third data points indicating the subject's analyte level at respective times within the first time-of-day range of a second day, and fourth data points indicating the subject's analyte level at respective times within the second time-of-day range of the second day,
the first time-of-day range is between a first time-of-day and a second time-of-day, the first time-of-day range is less than twenty-four hours,
the second time-of-day range is between a third time-of-day and a fourth time-of-day, the second time-of-day range is less than twenty-four hours, and
the first time-of-day range and the second time-of-day range are different;
determine a first time-in-target value indicating a percentage of time, within the first time-of-day range, that the subject's analyte level was less than a first threshold and greater than a second threshold using at least the first and third data points of the plurality of data points;
determine a second time-in-target value indicating a percentage of time, within the second time-of-day range, that the subject's analyte level was less than the first threshold and greater than the second threshold using at least the second and fourth data points of the plurality of data points; and
display the first time-in-target value and the second time-in-target value.

19. The system of claim 18, wherein the system is further configured to:
determine a first time-above-target value indicating a percentage of time, within the first time-of-day range, that the subject's analyte level was greater than a third threshold using at least the first and third data points of the plurality of data points;
determine a second time-above-target value indicating a percentage of time, within the second time-of-day range, that the subject's analyte level was greater than the third threshold using at least the second and fourth data points of the plurality of data points; and
display the first time-above-target value and the second time-above-target value.

20. The system of claim 19, wherein the third threshold is equal to the second threshold.

21. The system of claim 19, wherein the system is further configured to:
determine a first time-just-above-target value indicating a percentage of time, within the first time-of-day range, that the subject's analyte level was less than the third threshold and greater than the first threshold using at least the first and third data points of the plurality of data points;
determine a second time-just-above-target value indicating a percentage of time, within the second time-of-day range, that the subject's analyte level was less than the third threshold and greater than the first threshold using at least the second and fourth data points of the plurality of data points; and
display the first time-just-above-target value and the second time-just-above-target value.

22. The system of claim 18, wherein the plurality of data points includes at least fifth data points indicating the subject's analyte level at respective times within a third time-of-day range of the first day and sixth data points indicating the subject's analyte level at respective times within the third time-of-day range of the second day, the third time-of-day range is between a fifth time-of-day and a sixth time-of-day, the third time-of-day range is less than twenty-four hours, the first time-of-day range, the second time-of-day range, and the third time-of-day range are different, and the system is further configured to:
determine a third time-in-target value indicating a percentage of time, within the third time-of-day range, that the subject's analyte level was less than the first threshold and greater than the second threshold using at least the fifth and sixth data points of the plurality of data points; and
display the third time-in-target value.

23. The system of claim 18, wherein the system is further configured to determine, based on the plurality of data points, an analyte trend line indicating the subject's analyte level over time.

24. The system of claim 23, wherein the system is further configured to:
partition the analyte trend line into a plurality of segments, each segment representing the subject's analyte level over a discrete period of time;
for each respective segment, determine whether the segment indicates that the subject's analyte level was within a target range defined by the first threshold and the second threshold;
wherein determining the first time-in-target value comprises:
determining a first total time represented by the segments within the first time-of-day range that indicate that the subject's analyte level was within the target range; and
comparing the first total time to a second total time representing the time elapsed within the first time-of-day range over at least the first day and the second day.

25. The system of claim 24, wherein the system is further configured to associate each respective segment with a tag indicating the segment's determined status relative to the first and second thresholds.

26. The system of claim 24, wherein the system is further configured to:
receive user input to display a third time-in-target value indicating a percentage of time, within a third time-of-day range defined between a third time-of-day and a fourth time-of-day, that the subject's analyte level was within the target range, wherein the third time-of-day range is user-defined and different than the first and second time-of-day ranges;
in response to receiving the user input, determine the third time-in-target value by:
determining a third total time represented by the segments within the third time-of-day range that indicate that the subject's analyte level was within the target range; and
comparing the third total time to a fourth total time representing the time elapsed within the third time-of-day range over at least the first day and the second day; and
display the third time-in-target value.

27. The system of claim 18, wherein the first time-in-target value and the second time-in-target value are two time-in-target values among a plurality of time-in-target values, each time-in-target value of the plurality of time-in-target values indicating a respective percentage of time that the subject's analyte level was greater less than the first threshold and greater than the second threshold within a respective time-of-day range of a plurality of time-of-day ranges, the plurality of time-in-target values comprising at least three time-in-target values.

28. The system of claim 27, wherein each of the plurality of time-of-day ranges is non-overlapping and represents a time interval of eight hours or less.

29. The system of claim 27, wherein the plurality of time-of-day ranges comprises a range representing a morning period, a range representing midday period, a range representing an evening period, and a range representing a night period.

30. The system of claim 18, wherein the plurality of data points represent measurements collected over a sample period comprising at least seven days, and each of the determined time-in-target values reflects data collected from each day within the sample period.

31. The system of claim 24, wherein the step of partitioning the analyte trend line into a plurality of segments comprises:
determining a first point at which the trend line exceeds the first threshold and a second point at which the trend line falls below the first threshold; and
defining a segment as extending from the first point to the second point.

32. The system of claim 24, wherein the step of partitioning the analyte trend line into a plurality of segments comprises defining non-overlapping segments at regular time intervals.

33. The system of claim 18, wherein the analyte is glucose.

34. The system of claim 18, wherein the system is further configured to classify the data points of the plurality of data points into at least at least a first group for data points indicating the subject's analyte level at respective times within the first time-of-day range and a second group for data points indicating the subject's analyte level at respective times within the second time-of-day range.
